(19) United States
(12) Patent Application Publication
(10) Pub. No.: US 2011/0045093 A1
(43) Pub. Date: Feb. 24, 2011

(54) PHARMACEUTICAL COMPOSITION AND PROCESS COMPRISING VEGETABLE PROTEOLYTIC ENZYMES IN SUPRAMOLECULAR NANOPARTICLES, FOR THE TREATMENT OF PEYRONIE'S DISEASE, CONNECTIVE TISSUE DISEASES AND USE

(76) Inventor: Cristiano Alberto Ribeiro Santana, Sao Paulo (BR)

Correspondence Address: DUANE MORRIS LLP - Philadelphia;IP DEPARTMENT, 30 SOUTH 17TH STREET, PHILADELPHIA, PA, 19103-4196, US

(21) Appl. No.: 12/988,378

(22) PCT Filed: Apr. 13, 2009
(86) PCT No.: PCT/BR09/00107

§ 371 Date: Oct. 18, 2010

(30) Foreign Application Priority Data
May 13, 2008 (BR) PI0801929-0

(51) Int. Cl. A61K 38/48; A61K 9/14; A61P 19/00; A61P 15/10

(52) U.S. Cl. 424/491; 424/94.63; 424/94.65

(57) ABSTRACT

This invention refers to a pharmaceutical composition applied to the therapy of Peyronie's disease, in connective tissue diseases. The composition comprises, in its formulation, vegetable proteolytic enzymes encapsulated in the supramolecular nanoparticles.

This invention refers to a process and a new pharmaceutical applicable to any form, notably supramolecular nanoparticles, in the therapy of Peyronie's disease, in connective tissue diseases. The composition of this invention is the topical, non-toxic application with anti-inflammatory and debriding action, with strong penetration through skin.

Peyronie's disease has been known in medicine for over 270 years and is more particularly known in the area of urology.

Supramolecular Nanoparticles

Supramolecular nanoparticles are controlled release systems that biomimic endogen vectors of the organism, capable of encapsulating a variety of active agents (molecules: hydrophilic, hydrophobic, amphiphilic and macromolecules) for application in the pharmaceutical area. Such structures feature a spherical geometry and are made up of a solid, chemically modified starch nucleus, surrounded by a phospholipid bi-layer. Thus, the structure has a polar character in its interior and on the nanoparticle surface and an apolar one on the lipid bi-layer.

Such structures may be produced with the desired size, generally varying between 10 nm and 1000nm, depending on the application.

The system is dispersed in polar environments.

The structures are:
chemically, physical-chemically and mechanically stable (high temperature, increased shelf stability), in addition to being easily produced in industrial scale;
biocompatible, as they are composed by natural starch and lipids;
capable of stabilizing labile molecules, such as plant extracts and enzymes (e.g.: papain, bromelain and ficin—the biologic activity of which may be preserved for up to 18 months at 40° C.)
capable of efficiently penetrating the superior layers of the corneum.

Encapsulation Mechanism

The active agents may be incorporated and retained at the nanoparticles by means of chemical interactions. Two mechanisms may act in such molecule retention: ionic bridges and hydrophobic interactions.

### INVENTION DESCRIPTION

The skin permeability varies according to the body region, whereby the folds and face have the highest absorption. When a product is applied on the skin there will be a longer contact time and better percutaneous absorption.

According to the classic work “Histologia dos Epithelios” [Epithelial Histology] of Walter A. Hadler and Sineli R. Silveira, Publisher Editora Campus, Campinas, of 1993, it is considered that: “In view of the general morphological characteristics and the specialized functions that they perform, the epithelial cells are preliminarily classified in two categories, of which we use the supramolecular nanoparticle to protect and provide stability to the active agent, in this case papain/bromelain/ficin, as vegetable proteolytic enzymes. In this study, already evidenced, by means of the Franz cell, we may monitor, according to the nanoparticle size, the penetration depth of the active substance through the skin, affecting the area to be objectively treated. Another advantage is that the supramolecular particles are encapsulated and break at the area to be treated. For example, at the dermis or hypodermis. Another advantage is the use of less cream quantity, only at the area to be treated, that will penetrate by means of the epidermis corresponding to two epithelium classes: lining epithelial cells and secreting epithelial cells. The cells of such two categories are, together, the lining epithelia and the secreting epithelia, each one of them with specific peculiar functions. Such division is also based on the distribution of such two epithelium classes in the organism that, although wide, is different for both. In order to make up the lining epithelia, the epithelial cells interconnect side by side in such a way as to originate “membranes” or laminas superposed to the basal membrane the essential function of which consists of lining surfaces. In contrast, the secreting cells connect to build organized functional units, more adequate for the exercise of their specialized function, related to the secretion product synthesis; the secreting units are constituted this way. The lining epithelia are defined as living membranes, generally with discontinuity, that isolate the organism from the environment, by separating the internal medium from the external one. Moreover, such epithelia isolate, between each other, the several compartments of the internal medium, among which the intravascular compartment, the serous compartment and several others stand out. Among the multiple functions performed by the lining epithelia some are exercised by specialized varieties specifically adapted to the performance of one or more functions. Others are incorporated as general functions presented without distinction by the entire lining epithelial cell. The lining epithelial cell, like the majority of the living cells, passively absorbs water and electrolytes and actively eliminates them; such function is well developed in the epithelial cells. With this regard, an important exception shall be made: generally, absorption is understood as the solution penetration by means of the plasmatic cell membrane. However, two specific absorption forms shall be distinguished: passive absorption that follows the osmosis laws, and active absorption that entails the effective participation of the epithelial cell and does not follow such physical laws. On the other hand, it must be considered that every substance needs, in order to penetrate to the interior of a multi-cell organism, or to be excreted or eliminated, to cross at least one lining epithelium, as every higher organism superior is penetrated externally and internally by epithelia. It should also taken into account that the lining epithelia, although they continuously recover and protect the surfaces they line, are not impermeable; therefore, they do not behave as inert “membranes”. In contrast, they allow the exchange of gases, water, of several electrolyte types and of certain other solutes between the internal and external environments, or among the several internal compartments, which characterizes their permeability. The lining epithelial cells limit, in a controlled and selective fashion, the permeability of the respective epithelia, in order to protect the organism without stopping controlling the homeostasis. In order to fulfill such function, the epithelia organize themselves and use their cells in a special way with the intention of constituting linings the cells of which are juxtaposed to the basal membrane and are interconnected by means of intercellular junctions; on their part, the cells are lined by the plasmatic membrane, with peculiar characteristics, and by the glycocalyx, both capable of expressing well defined functional properties. The functional characteristics expressed by the part of the plasmatic membrane that lines the apical cell surface are different from those expressed by the part located on the basal or basolateral front; such differences that foremost occur from a functional point of view contribute to the increased polarization degree expressed by the lining epithelial cells. The fundamental function performed by the lining epithelia, essentially consists of the protection with which they provide the surface they line, characterizing their protecting lining function. Such function features a special characteristic, as it is a lining that, in addition to mechanically, physically and chemically protecting the lined surface, is not inert. The lining epithelia are permeable, which allows for a controlled and selective passageway of several products through its wall. There are many evidences in favor of the understanding that the permeability of the lining epithelia constitutes a fundamental property, with significant functional expression, since it is essential for the exercise of several functions performed by the epithelia, especially as it is selective and its permeability degree features an extensive variation. It is well proven that the permeability degree strongly effects the function executed by the lining epithelia: 1) wide permeability; 2) reduced permeability and 3) no permeability. In the event of wide permeability, the epithelia allow for intense metabolic exchange through their walls, and the control and selectivity of its permeability is low. In such circumstances, the epithelium acts on the filtration and transfer of metabolites, and such functions require low qualitative control; the exercise of such functions is subordinate to the intrinsic epithelium structure that is adapted to act, foremost passively, with a low selective permeability level. The lining epithelia the permeability degree of which is reduced, due to their peculiar characteristics, feature the property to partially control their own permeability, foremost the selectivity. As a consequence, such lining epithelia present selective permeability, which allows them to interfere and qualitatively control their functional activity, as well as it provides them with more aptness to act on the homeostasis control. The absence of such permeability of the epithelium is correlated with the complex isolation of the lined surface and, on the other hand, with more functional control of such epithelium, as its cells, although very permeable, have selective permeability. In that case, the lined surface is delimited by an impermeable or little permeable and very effective “membrane” that executes the important protection function as it is able to differentiate what may pass through the epithelium. The permeability of the lining epithelia is such an expressive functional property that it has been used as important criterion to classify it in three classes: 1) permeable epithelia; 2) little permeable epithelia and impermeable epithelia. Due to its selective permeability, even in lower animals the epithelia assume the function of recovering the organism, constituting the external lining, with a limiting and protecting property, not only morphological, but also functional. The cells, basically little differentiated, behaved as a little effective semi-permeable “membrane” that acted passively, but the function of which allowed for the separation, although more primitive and more morphological than functional, between the internal and external environments. It seems that the majority of the lining epithelia acts as barrier that impedes free passive diffusion, as its permeability, that is selective, is conditioned to several factors among which the electric potential in the plasmatic membrane of its cells should be stressed. The continuity of the epithelial lining is established both through the intimate juxtaposition of the adjacent cells and through the presence of intercellular connection devices. The epithelial cells are involved by the glycocalyx that also takes part in the lining function performed by the epithelium, in addition to contributing to the connection between adjacent cells, as the intercellular cement is also made by the glycocalyx. Several experimental investigations confirm that the selective permeability of the lining epithelia is associated to other specific functions exercised by their cells, namely: absorption, excretion and secretion. Such functions are, by superposing the permeability which is the fundamental function, responsible for the general functioning of the epithelial cell. Basically, the following general functions are performed by the lining epithelia: 1) protective surface lining function; 2) functional individualization and isolation of the internal environment and of the difference compartments, due to the selective permeability of its cells; 3) homeostasis control of the internal environment due to the aptness of the cells to interfere in the selective permeability of the epithelium; The epithelial cells express their capacity to perform the absorption, secretion and excretion; such functions interfere with the permeability of the epithelium; 4) performance of metabolic functions due to their aptness to carry out sodium and water exchanges and perform the metabolite transfer due to the high and little selective permeability degree of the cells and the intercellular space; 5) product transport along the epithelial surface due to the participation of cilia; 6) sensorial perception and 7) germinal function. Among its functions, the four first ones stem foremost from the selective permeability of the epithelial cells, over which the additional effects regarding its absorption, excretion and secretion properties superpose. Among the general functions performed by the lining epithelia, the selective permeability is responsible for the efficiency with regard to the capacity of lining, protecting and isolating the surfaces, as well as the capacity of controlling the homeostasis; the passive absorption and metabolite transfer capacity are normally carried out by the majority of the cells of such epithelia that require only small adaptations in order to become able for the effective exercise of such functions. In contrast, the absorption, excretion and secretion functions depend on properties that are successively developed and would become preponderant, particularly in some specialized types of lining epithelia that have adapted by following a new and specific direction. The sensorial perception and its germinal function are the most specific functions only expressed by certain even more specialized epithelia. Considering the morphological characteristics of its cells, the lining epithelia have been classified according to the same number of cellular strata that they have into: simple (single stratum) and stratified (two or more strata). Both simple and stratified epithelia are on their part, according to their cell form, divided into squamous, cubic or prismatic. The simple epithelia are generally adapted to fully express their most expressive fundamental property that consists of their permeability, the selectivity degree of which varies. The simple lining epithelia, constituted of a single squamous or cubic-prismatic cell layer, present big differences with regard to their functional properties correlated not only to the morphology of their cells, but also to the properties of the intercellular space. The squamous simple epithelia are generally quite permeable; the cubic-prismatic epithelia are less permeable. The permeability of the lining epithelia is not only selective, but also controlled by the functional activity of their cells, although the control is the less effective the higher the permeability of the intercellular space. The cubic-prismatic epithelia control, because they are less permeable than the squamous ones, their permeability more efficiently. Based on the form of the epithelial cell, on its permeability and on the most common adaptations that the lining epithelia present, a provisional classification of such epithelia may be carried out. Thus, the simple lining epithelia are divided into two categories: squamous and cubic-prismatic. Each category is subdivided, according to its functional properties, into open or permeable epithelia, into semi-occlusive or little permeable epithelia and into occlusive or impermeable epithelia. In the classification of the simple lining epithelia, generally the different cubic and prismatic epithelia are considered that are defined and identified based on the form of the epithelial cells that constitute them, however functional studies have shown that the correlation between form and function has many exceptions. Therefore, a functional classification is adopted, mainly considering its permeability. According to that criterion, those epithelia are referred to as cubic-prismatic including the semi-occlusive and occlusive epithelia. According to the same criterion, the stratified epithelia may be sub-divided into: squamous and cubic-prismatic. The stratified epithelia are adapted to exercise foremost the mechanical protection function, as they are impermeable or little permeable. The epithelia comprise, in addition to the cells, the intercellular space and the basal membrane that interfere with the permeability degree; such permeability does not only depend on the peculiar properties of their cells, responsible for the transcellular permeability pathway, but also on the presence of another permeability pathway on the walls, constituting the intercellular or paracellular pathway. The transcellular pathway comprises two different pathways that consist of the transmembraneous pathway and of the transcanalicular pathway or transcytosis. It has been proven in experiments that the lining epithelia may be transposed by water and by substances of different natures, both by means of their epithelial cells (transcellular pathway) and by the pathway located between their cells (intercellular pathway). In the first case, the epithelial cell may perform the permeability control of the epithelium by means of its biological activity, making such process selective. With regard to the permeability of the intercellular space, the epithelial cell, although not behaving completely passively, does not directly interfere with the transport. The only active participation of the cell consists, in this case, exceptionally in determining the extension of the corresponding intercellular space. By means of an action of the microfilaments that constitute its cytoskeleton on the epithelial cell, foremost certain types of simple squamous open lining epithelia, it may change the form and retract segments form its cytoplasm; consequently, it may act upon the amplitude of the intercellular space by regulating it. It has been established that the transcellular permeability of the simple lining epithelia is perfectly different from the intercellular permeability, as the two are subordinated to very different mechanisms. The permeability of the epithelial cell that is selective is subject to the influence of its biological activity; in contrast, the intercellular permeability is totally passive and, therefore, not selective.

By means of researches carried out by the applicant of this invention, among which animal experiments rarely carried out stand out that assess the histological and morphometric aspect of the tissue repair by means of the application of the now invented composition with the use of papain and bromelain. In such studies, 40 adult rats (RATTUS NORVEGICUS ALBINUS) were used that were kept in isolated cages with food and water. The animals were distributed into two groups of 20 animals each, depending on the type of treatment, i.e. one group of 20 animals in which no type of treatment was performed and another group of 20 animals that were treated with a 2% papain solution in topical use at the wound.

The animals were treated and anesthetized for the due procedures and the histological cuts were analyzed under a normal optical microscope, and the results were submitted to comparative analysis, in both groups according to the fibroblast and collagen fiber dates.

Other researches were carried out in which, for example, 24 men between 41 and 72 years of age, all of white skin color, were analyzed in 63 cases, all affected by Peyronie's disease to different degrees, divided into three groups:
I. Fibrosis in a small different region of the penis without curvature deviation;
II. Longitudinal fibroses or fibrosis at area 15 circumscribed with penis deviation: (curvature):
III. Fibrosis with calcified plates with a deviation upon penis erection.

The patients of group II had the following results: 60% full recovery of the wound, 20% partial recovery and 20% no recovery.

Such researches were assessed, and it was concluded that in the inflammation process there are several entailed factors, among them foremost the vasodilatation with exudation; migration leucocytes and macrophages; proliferation of fibroblasts; action of chemical mediators such as histamine, serotonin, bradykinin and prostaglandins; proteolytic activity.

With regard to the activity, it refers to the involvement of the proteases (chymotrypsin and cathepsin) and in the collagenase that currently does not only facilitate the destruction of foreign bodies, but also hydrolyzes the collagen the resulting peptides of which act as chemotactic substance stimulating the fibroblast proliferation.

When observing the wounds medicated with the composition according to this invention comprising papain and bromelain, it is perceived that the granulation tissue is more developed with a larger number of fibroblasts and collagen fibers, in view of the fact that the mentioned composition may have assisted also in the digestion of the denatured collagen. According to what has been observed in this experiment, the papain solution acted similarly to the other proteases, i.e. digesting tissue rests of proteic nature that result in peptides that are chemotactic for fibroblasts, prematurely stimulating fibroplasia in the group medicated with the 2% papain solution it caused the more effective tissue repair in the superficial and deep region of the fibrosis and kelloid lesions and is, therefore, an effective and non-invasive alternative for the treatment of Peyronie's disease.

Thus, in order to obtain the results mentioned above, the applicant developed, by means of material of the composition a therapeutic method that, in a period of 8 to 12 months, were applied daily on the penis, consisting of: application of this formulation for a duration of 30 minutes without removal in order to arrange for penetration; this application was done in the dorsal and lateral regions up to the pendular region, and no application at the gland is required.

Thus, in order to obtain a formulation of topical use with high absorption through the skin that evidentially obtains an improvement of 90% to 96% of the disease affection, this composition was created applicable to the therapy of fibrotic pathologies and diseases activating the action of the proteolytic enzymes with debridant and anti-inflammatory activities in the repair of fibrotic lesions.

Microcirculatory Unit

The microcirculatory unit, with a gyrating plate of the cell life, is a tissue balance center, therefore, the different vascular systems need to adapt to the circulatory variations.

When the venous disturbance compensation mechanisms are overcome, the vascular and tissue structures are altered.

The venous stasis leads to an increase in the intracapillary pressure. The capillary permeability is increased, which translated into the evasion of liquids and proteins with high molecular weight to the connective tissue.

The permeability excess and interstitial inundation are the origin of lymphatic overload with edema installation.

The release of aggressive substances, such as histamine, serotonin, prostaglandins, cause a series of tissue reactions. If the protein excesses are not depolymerized by the macrophages, a fibroblastic stimulation and fibroses installation occurs that, on its part, maintains and aggravates the venocapillary-lymphatic stasis closing the pathological cycle.

Diffusion Cells-Nanoparticle Penetration Monitoring

The in vitro techniques require relatively sensible experimental conditions and allow for high-precision analytical sampling.

The experimental use of the in vitro methods allows for the use of diffusion cells. Such cells may have different designs. The vertical diffusion cell was popularized by Dr. Thomas Franz and is therefore known as Franz cell. This diffusion cell model has been applied to various cutaneous permeation studies, including studies of formulations for the topical drug release, as well as ophthalmic, cosmetic products, products for skin care and pesticides.

According to the IUPAC 88 guide of the FDA, the vertical diffusion cell is an ideal quality control tool for topical preparations. The glass cell set includes: receptor chamber, supplying chamber with lock, openings for sampling and medium reception, external jacket for temperature control, helix agitator and dosage ring for infinite dosage.

The pig ears are flayed at the slaughterhouse without boiling them. The ears are cleaned and the entire skin is separated from the fat. After stabilizing the temperature, the cells are opened and assembled with the skins on which the products were applied, so that the depth to be reached by the active substance contained in the nanoparticles may be monitored according to the size of the nanoparticles.

Adipose Cellulitic Tissue

The adipose tissue develops slowly until becoming cellulite in four successive phases:

Interstitial Edema: consequence of venous stasis and of excessive capillary permeability, there is a capillary distension, increase of the liquids passing through and formation of edemas in the sinus of the connective tissue with lymphatic overload. The adipocytes become hypertrophic and connect to each other forming a block.

Formation of a Connective Net: the physical-chemical modifications cause the formation of a net that infiltrates into scleroialine bands around the fat masses.

Formation of Micronodi, then Macronodi: the adipose masses group in closed micronodi in the connective fiber and end up forming macronodi identified by palpation.

Capillary Alterations: the same ones that are normally observed in the development of varices; ectasias, aneurysm, thickening of the basal layer.

Through researches carried out by the applicant of this invention, experiments in animals and humans stand out. The animals selected were rats (Adults Rattus Norvegicus Albinus).

Cuts were analyzes in a normal optical microscope, and the results were submitted to comparable analyses in all groups, in accordance with the classification of fibroblasts, collagen fibers and leucocytes in tables specifically designed for such purpose. In adults, 21 women with multiple hypertrophic scar lesions, keloids and vasculopathic dermopanniculosis (Cellulitis) were examined. 06 cases of hypertrophic scars, 15 cases of cellulitis, and 04 cases of Dupuytren's disease were distributed.

Cellulitis Types

| | |
|---|---|
| MILD CELLULITIS | 50% |
| COMPACT CELLULITIS | 20% |
| DERMATOUS CELLULITIS | 30% |

Studied Age Range:

| | |
|---|---|
| 15 TO 25 YEARS | 40% |
| 26 TO 35 YEARS | 30% |
| 36 TO 45 YEARS | 19% |
| 46 TO 50 YEARS | 10% |
| 56 TO 65 YEARS | 06% |

Hypertrophic Scar

In the 4 cases, two daily applications.

The improvement estimated after 60 days is 50%, whereby basically the depigmentation and reduction of the scar height occurs. After such period, the fibrotic part of the lesion softens with a tendency of a reepithelization of the affected area.

Dupuytren's Disease

The studies were carried out in the region of the palm in simple or multiple lesions, with a treatment of 1 hand or 2 hands simultaneously. The treatment involves running the gel twice daily at the area of the lesion. Improvement including absence of pain is estimated after 30 days by 40%.

After such period, the fibrotic hardened part (plate) starts softening.

The subject-matter of this Invention is a new “PHARMACEUTICAL COMPOSITION”, and such composition contains papain at more than 10 nm for 100 g of cream.

Advantageously this invention may comprise the following formulation:
PAPAIN . . . between 10 nm and 1,000 nm

Advantageously this invention may also comprise the following formulation:
BROMELAIN . . . between 10 nm and 1,000 nm

Even more advantageously this invention may comprise the following formulation:
FICIN . . . between 10 nm and 1,000 nm

1. “PHARMACEUTICAL PROCESS COMPRISING VEGETABLE PROTEOLYTIC ENZYMES IN SUPRAMOLECULAR NANOPARTICLES, FOR THE TREATMENT OF PEYRONIE'S DISEASE AND CONNECTIVE TISSUE DISEASES”, characterized by the fact that the process comprises structures that have a spherical geometry, made up of a solid chemically modified starch nucleus, surrounded by a phospholipid bi-layer; the structure has a polar character in its interior and on the nanoparticle surface and an apolar character on the lipidic bi-layer, whereby the structures produced with sizes varying between 10 nm and 1000 nm and the active agents present in the composition are incorporated and retained in the nanoparticles by means of chemical interactions, with the use of two mechanisms that may act in such molecule retention: ionic bridges and hydrophobic interactions; and the process also comprises the monitoring phase according to the nanoparticle size, the penetration depth of the active substance through the skin affecting the area to be treated, whereby the supramolecular particles are encapsulated and break at the area to be treated.

2. “PHARMACEUTICAL COMPOSITION COMPRISING VEGETABLE PROTEOLYTIC ENZYMES IN SUPRAMOLECULAR NANOPARTICLES, FOR THE TREATMENT OF PEYRONIE'S DISEASE AND CONNECTIVE TISSUE DISEASES” characterized by the fact of comprising in its formulation:
PAPAIN . . . between 10 nm and 1,000 nm.

3. “PHARMACEUTICAL COMPOSITION” characterized by the fact of comprising:
BROMELAIN . . . between 10 nm and 1,000 nm.

4. “PHARMACEUTICAL COMPOSITION”, according to claim 2, characterized by the fact of comprising:
FICIN . . . between 10 nm and 1,000 nm.

5. “PHARMACEUTICAL COMPOSITION”, according to any of claim 2, 3 or 4 characterized by the fact that it is presented in the form of supramolecular nanoparticles.

6. “USE OF THE PHARMACEUTICAL COMPOSITION”, according to any of claims 2 to 4, characterized by the fact that it is employed for the production of a drug against the most different diseases, notably against Peyronie's disease and against connective tissue diseases.
